(19) United States
(12) Patent Application Publication
(10) Pub. No.: US 2026/0108406 A1
(43) Pub. Date: Apr. 23, 2026

(54) Multi-Functional Patient Positioning, Warming, and Lateral Transfer Device for Surgical Safety

(71) Applicant: BRANDON CUONGQUOC GIAP, Laguna Niguel, CA (US)

(72) Inventor: BRANDON CUONGQUOC GIAP, Laguna Niguel, CA (US)

(21) Appl. No.: 18/919,202

(22) Filed: Oct. 17, 2024

(51) Int. Cl. A61G 7/10; A61F 7/00; A61G 7/057

(52) U.S. Cl. CPC A61G 7/1026; A61F 7/0097; A61G 7/05769; A61G 7/1028; A61F 2007/006; A61F 2007/0091

(57) ABSTRACT

The invention improves air-assisted lateral transfer devices by integrating elevated strips along the sides of the air mattress. These strips, ranging from 0.5 mm to 5 mm in thickness, limit lateral air dissipation, enhancing the stability and effectiveness of the air cushion. This advancement improves patient transfer efficiency, safety, and operational performance in medical and surgical settings.

### CROSS-REFERENCE TO RELATED APPLICATIONS

This application is a continuation-in-part of Ser. No. 18/919,202, filed on Oct. 17, 2024, titled “Multi-Functional Patient Positioning, Warming, and Lateral Transfer Device for Surgical Safety,” the entirety of which is incorporated herein by reference.

### FIELD OF THE INVENTION

The present invention relates to medical devices, specifically to patient positioning, warming, and lateral transfer devices used in surgical and medical environments. This invention incorporates an improvement to air-assisted transfer systems by introducing elevated strips to enhance air retention beneath the device, facilitating smoother patient transfers.

### BACKGROUND OF THE INVENTION

Patient handling during surgical and medical procedures requires devices that can address positioning, temperature regulation, and efficient lateral transfer. Over the years, advancements in air-assisted lateral transfer devices have provided significant benefits by reducing the friction between the patient and the surface during transfers. These systems typically employ an inflatable air mattress to create a cushion of air, enabling the patient to “hover”slightly above the surface for smooth movement.

Despite their utility, these devices face critical challenges. One of the primary issues is the rapid dissipation of escaped air. When air escapes laterally from the mattress, it diminishes the effectiveness of the air cushion, often necessitating higher air flow rates to maintain operational efficiency. This not only increases energy consumption but also creates noise and disrupts laminar airflow in sterile environments like operating rooms. Furthermore, the loss of air cushion stability can lead to uneven transfer processes, posing risks to both patients and medical staff.

Another limitation is the difficulty in maintaining a robust air cushion when handling heavier patients or performing lateral transfers over longer distances. Existing solutions fail to address how to effectively contain escaped air to ensure consistent performance.

To overcome these shortcomings, the present invention introduces elevated strips or variations thereof along the sides of the air mattress. These strips act as barriers, reducing lateral air escape and retaining more of the air beneath the mattress. This design innovation maintains a stable air cushion for longer periods, improving the safety and efficiency of patient transfers. Additionally, the low profile of these strips ensures they do not interfere with patient positioning or the inflation process.

By addressing these critical gaps, the invention significantly enhances the functionality of air-assisted transfer devices, making them more reliable and efficient in diverse medical settings.

### SUMMARY OF THE INVENTION

The present invention is a multi-functional patient positioning, warming, and lateral transfer device designed to enhance patient safety and comfort during surgical procedures. This invention addresses several key challenges faced in the operating room, including patient positioning and protection, body temperature regulation, and efficient post-surgery transfer. It integrates multiple functions into a single, comprehensive device that improves patient care while streamlining workflows for medical staff.

In an embodiment of the present invention, the device incorporates flexible, padded substrates on its top surface, which can wrap around the patient's limbs to create a secure and protective engagement. This wrapped engagement prevents injury to vulnerable areas like the ulnar nerves, protecting the patient from pressure injuries that can occur during surgery. The padded layers slightly elevate the patient's arms, further reducing the risk of nerve compression and other pressure-related injuries.

Additionally, the flexible substrates serve to secure the patient to the surgical table, preventing unwanted movement or sliding, particularly for anesthetized patients. Multiple hook-and-loop Velcro fasteners, webbing straps, and buckles ensure the patient remains safely positioned throughout the surgery.

A three-layer temperature-regulating pad is incorporated into the device, which works in conjunction with a forced-air warming system to maintain the patient's body temperature during surgery. The warming system consists of:
A top air-permeable layer that allows warm air to pass through to the patient's body space.
A middle layer with engineered perforations (1 mm diameter holes) to optimize warm air distribution across the patient's head, arms, and legs.
A bottom layer that helps circulate warm air and directs it through the middle layer, ensuring efficient heat transfer and minimizing heat loss around the edges.

The pad's design ensures that warm air is evenly distributed across the patient's body, preventing hot spots and providing a consistent cushion of warm air, which helps maintain normothermia throughout the procedure.

The device also features inflatable support for easy lateral transfer of the patient after surgery. The bottom two layers of the mat are designed for this function since the fourth layer (without holes) and the fifth layer (with holes) create an air cushion beneath the patient. Upon connecting a high-flow air machine, the mattress inflates, slightly elevating the patient, while air escapes through small holes in the bottom layer, reducing friction between the mat and the surgical table. 3

This system, often referred to as hover technology, enables frictionless movement, allowing the medical team to easily slide the patient onto a transport bed without manual lifting, reducing the risk of injury to both the patient and medical staff.

The invention includes several anti-slip mechanisms to prevent displacement of both the patient and the pad during surgery or lateral transfer. Non-slip strips integrated into the pad's bottom layer prevent movement when the pad is deflated. These strips do not interfere with lateral transfer since they are neutralized by the air cushion generated during inflation. Additionally, fastening mechanisms, including straps and buckles, secure the device to the surgical table, preventing sliding when the table is adjusted (e.g., during head-down or lateral-tilt positions).

The mat is designed to create a trough-like effect during inflation, where the sides inflate higher than the center, effectively cradling the patient in place. Combined with the arm wraps and leg fasteners, this design ensures the patient remains securely positioned, minimizing the risk of movement during inflation and transfer.

To address the challenge of securing critical medical lines during surgery, the device incorporates strategically placed Velcro straps and webbing at key locations. These fasteners allow medical lines and tubes (e.g., IVs, catheters) to be safely secured, reducing the risk of dislodgement or tangling during the procedure or patient transfer.

The top layer is an air-permeable, non-woven polypropylene material that allows soft, warm air to flow through to the patient's body space while preventing fluid penetration. The second layer is engineered with perforations for optimal air distribution. The third layer serves as the base of the warming system, helping to direct air flow through the perforated layer. The fourth layer forms part of the inflatable support system for patient transfer, without perforations to contain air for inflation. The fifth layer contains small holes that allow air to escape and create a cushion beneath the pad, reducing friction during lateral transfer.

The air inlet for the warming system is designed with an elastic band to fit various air hoses, allowing compatibility with different forced-air warming machines. The warming blanket is sealed around its edges using either double-sewn lines or heat-sealing technology to prevent heat loss and ensure efficient air distribution.

This continuation-in-part introduces two or more slightly elevated strips along the sides of the air mattress. These strips, constructed from durable materials such as rubber or thick tape or integrally formed by sewn folds in the fabric during manufacturing or equivalent methods, range in thickness from 0.5 mm to 5 mm and are positioned longitudinally to retain escaped air beneath the mattress. This continuation-in-part introduces two or more slightly elevated strips along the sides of the air mattress. These strips, constructed from durable materials such as rubber or thick tape or integrally formed by sewn folds in the fabric during manufacturing or equivalent methods, range in thickness from 0.5 mm to 5 mm and are positioned longitudinally to retain escaped air beneath the mattress. The low profile ensures the strips do not interfere with patient positioning or the inflation process, while their durable construction ensures long-term reliability. By limiting air escape, the elevated strips reduce the need for high-flow air machines, conserving energy and minimizing operational noise. This enhancement ensures a consistent air cushion, even when transferring heavier patients or over longer distances. The retention of escaped air beneath the mattress prevents uneven deflation, reducing the risk of patient sliding or instability during transfer. The improved air retention reduces the demand for high airflow, ensuring quieter operation and maintaining laminar airflow in sterile environments like operating rooms.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE ACCOMPANYING DRAWINGS

The aforesaid as well as other objects and advantages of the invention will appear hereinafter from the following description taken in connection with the accompanying drawings in which:

FIGS. 1A and 1B illustrate the structure of the top three layers of the multi-functional patient positioning device, showcasing their respective roles in patient warming and positionings;

FIGS. 2A and 2B depict the warming functionality of the top three layers, focusing on the distribution of warm air across the patient's body during surgical procedures;

FIG. 3A to 3C demonstrate how warm air flow is controlled and distributed by the top three layers, ensuring even heat distribution without hotspots;

FIG. 4 shows the inflatable support mechanism used during lateral patient transfer, emphasizing the creation of an air cushion for frictionless movement;

FIG. 5 illustrates the working of warming function wherein flow is controlled by top three layers according to an embodiment of the invention;

FIG. 6 provides a detailed view of the fastening mechanisms, including Velcro straps, buckles, and non-slip strips, that secure the device to the surgical table and the patient to the device;

FIG. 7 illustrates the strategic placement of Velcro straps and webbing to secure medical lines and tubes, minimizing the risk of tangling or dislodgement during use;

FIGS. 8A and 8B shows the design and operation of the air inlet system, compatible with various forced-air warming machines and designed for efficient airflow management;

FIG. 9A to 9C depict the deflated and inflated states of the device, illustrating the transition and function of the air cushion during lateral patient transfer;

FIG. 10A to 10D show the multi-layer construction of the device, focusing on how each layer contributes to warming, patient positioning, and transfer;

FIG. 11 highlights the cross-sectional view of the air-retention strips and their integration into the mattress design to enhance energy efficiency and noise reduction; and

FIG. 12 summarizes the integrated functionality of the device, combining warming, positioning, and transfer mechanisms into a cohesive system.

### DETAILED DESCRIPTION OF THE INVENTION

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

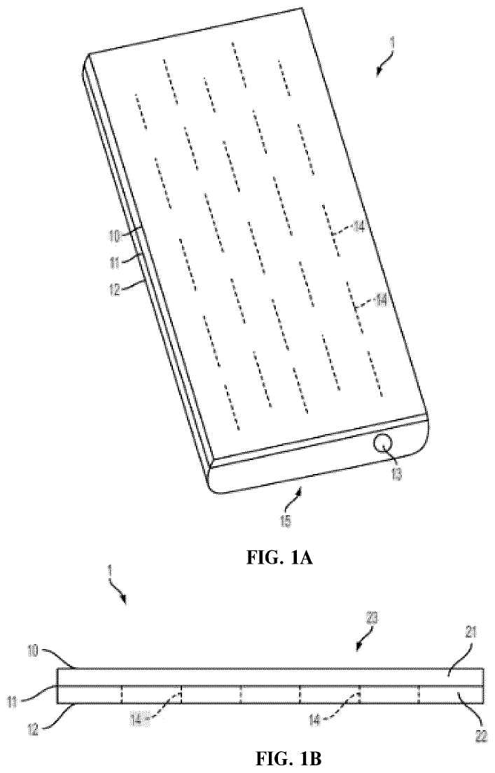

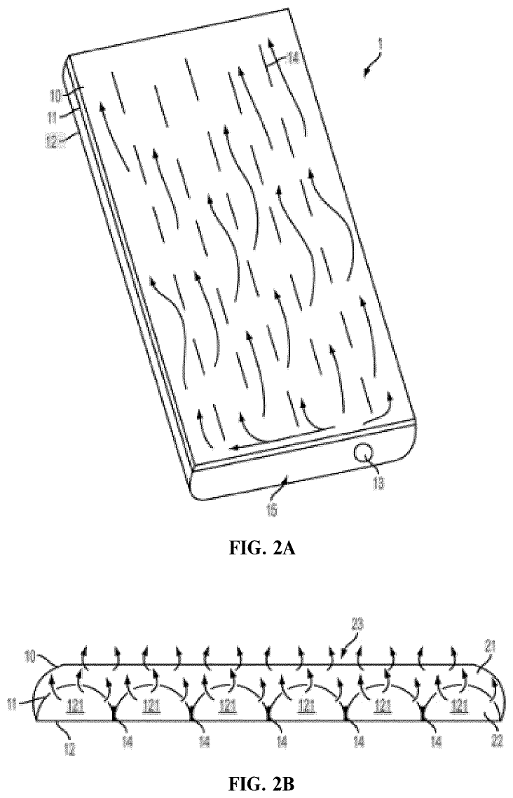

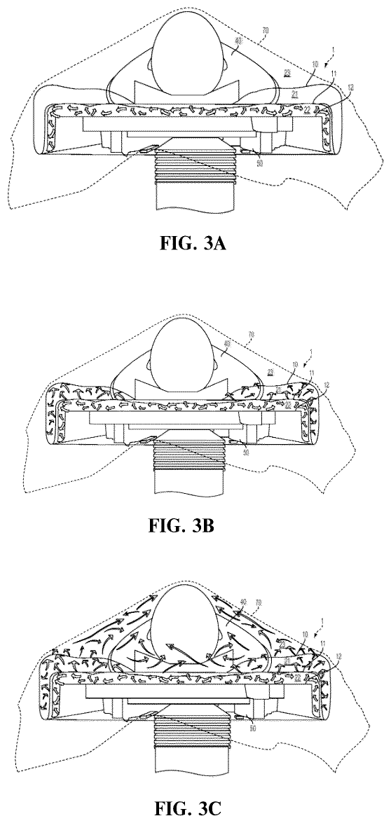

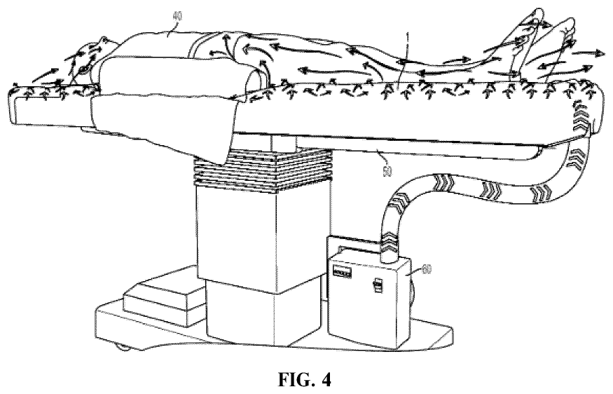

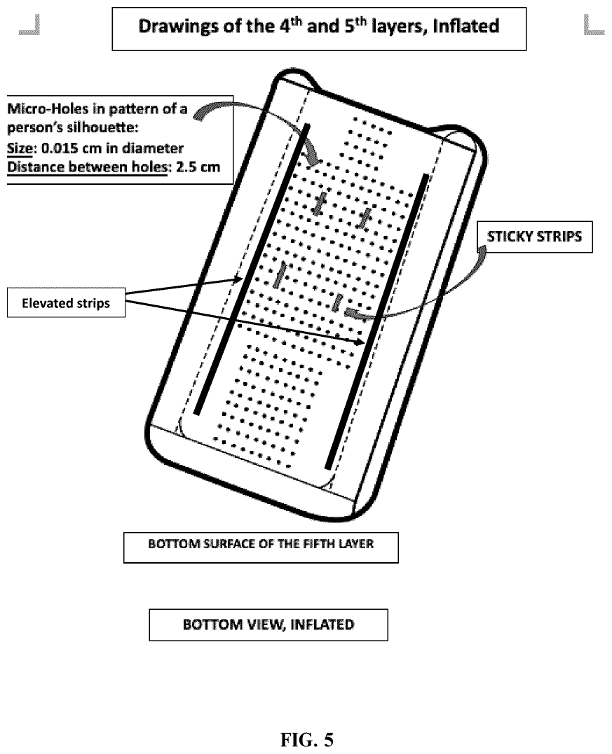

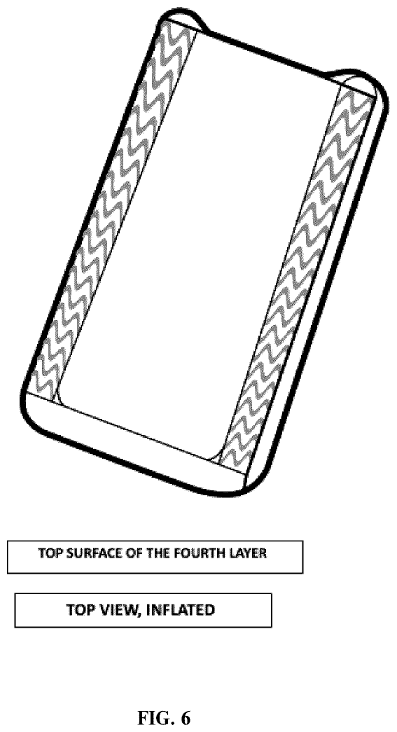

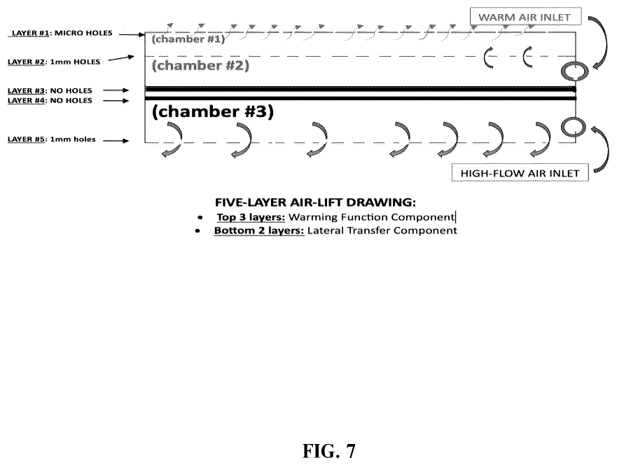

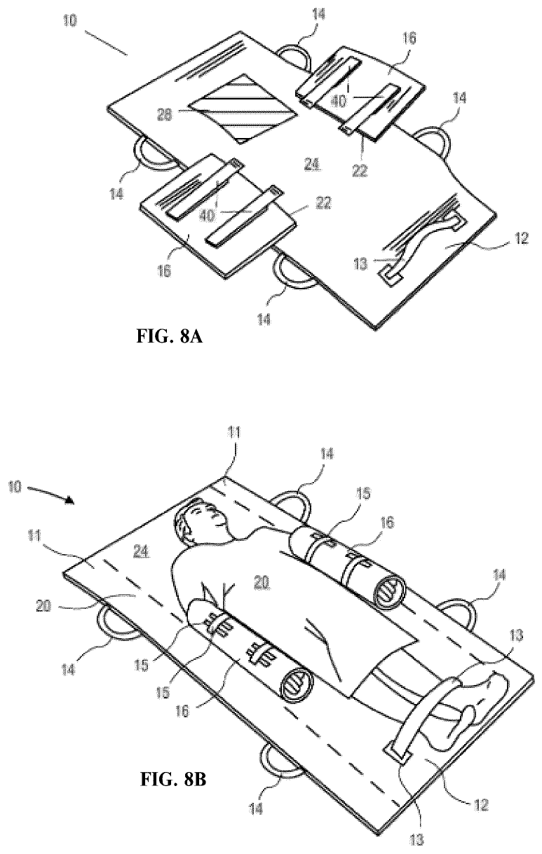

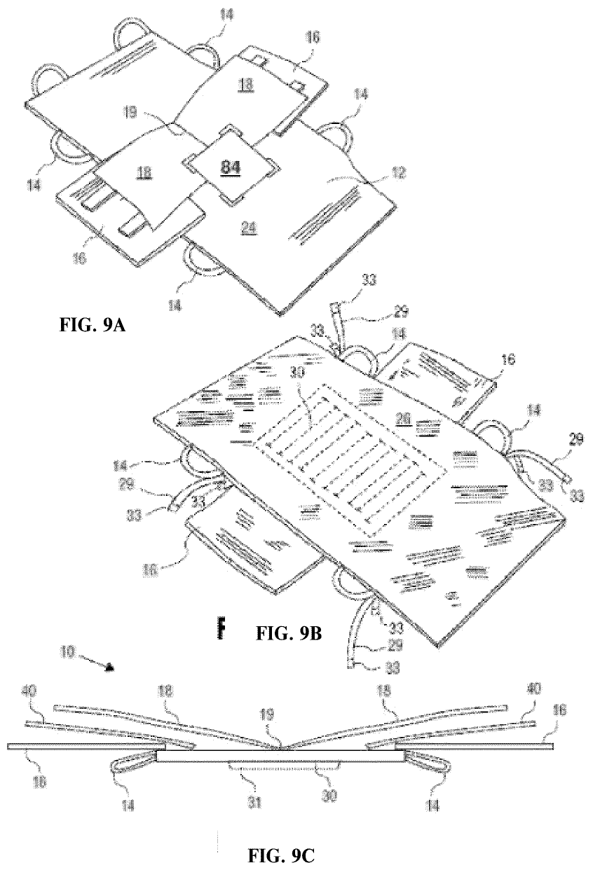

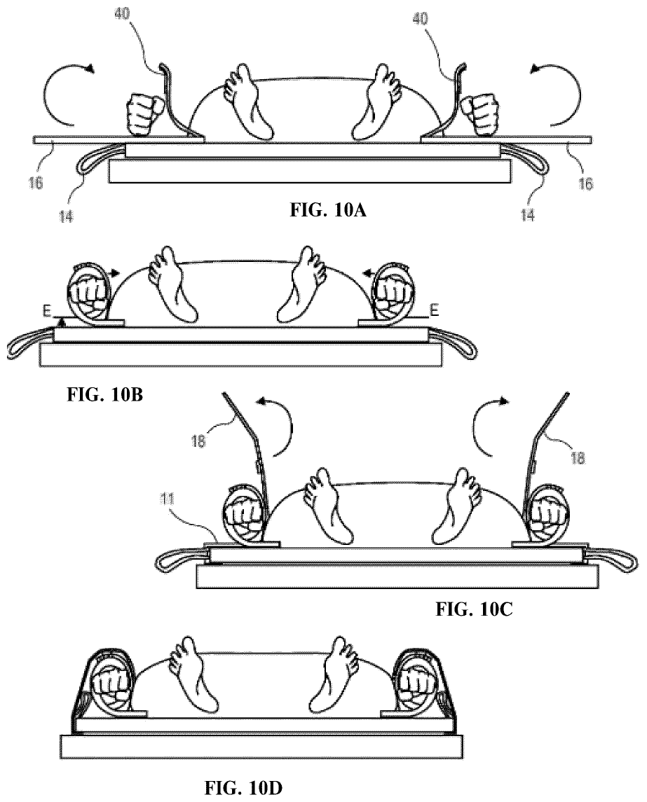

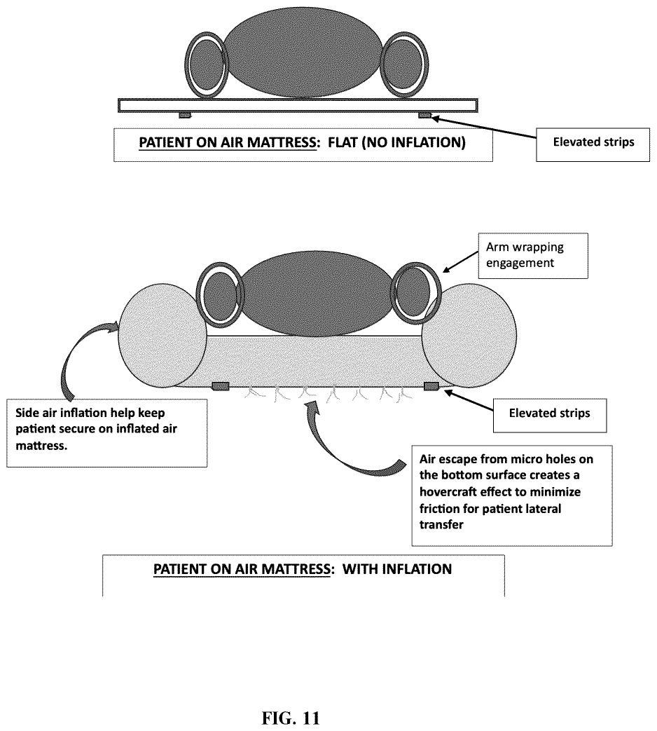

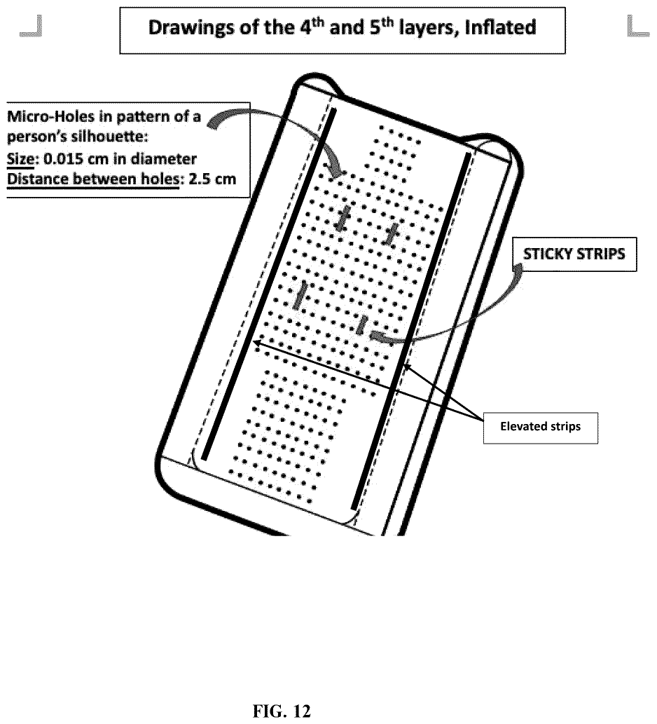

With reference now to the drawings, and in particular from FIG. 1A to FIG. 12 thereof, Multi-Functional Patient Positioning, Warming, and Enhanced Lateral Transfer Device with Elevated Air Retention Strips embodying the principles and concepts of the present invention is described.

The present invention is a multi-functional patient positioning, warming, and lateral transfer device designed to enhance patient safety and comfort during surgical procedures. This device integrates multiple features into a single comprehensive system, addressing challenges in patient handling, protection, and temperature regulation during surgery, and offering a safe method for transferring patients afterward.

In an embodiment of the present invention, the device consists of five distinct layers, each designed to perform specific functions. The layers are bonded together using either sewing or adhesive methods, ensuring structural integrity during use. Additionally, the mat can be secured to the surgical table using various fastening mechanisms such as straps, buckles, or hook-and-loop fasteners (Velcro). These securement mechanisms help prevent unwanted movement of the patient or device during surgery or lateral transfer.

The top layer of the device is made of an air-permeable, non-woven polypropylene fabric. This layer is fluid-resistant to prevent liquids (such as blood or saline) from permeating through to the lower layers of the device, protecting the warming system and maintaining a sterile surgical environment. The top layer also allows warm air to pass through its micro-perforations, creating a soft air cushion around the patient, which helps in regulating the patient's body temperature.

The second layer is a perforated middle layer, engineered with 1 mm diameter holes spaced approximately 2.5 cm apart. These perforations are strategically positioned around areas that require optimal warming, such as the patient's head, arms, and legs. This design ensures an even and efficient distribution of warm air, maximizing the warming effect in critical areas.

The third layer serves as the base for the warming system. This layer works in tandem with the second layer to direct warm air upward toward the patient. Air is introduced into the system through an air inlet, typically located near the sides or corners of the warming section. The air inlet is designed to accommodate various air hoses from different forced-air warming machines, ensuring the device is compatible with multiple warming systems. Elastic bands around the air inlet allow for a snug, secure fit.

The fourth layer is part of the lateral transfer system and serves as one of the air chambers for the inflatable support feature. This layer is designed without perforations to retain air when the mattress is inflated. The inflation process slightly elevates the patient, reducing the friction between the pad and the surgical table surface. The ability to inflate the fourth layer aids in patient transfer by creating a cushion of air beneath the patient, allowing for easy sliding from the surgical table to a transport bed.

The fifth layer, located at the bottom of the device, features small perforations designed to release air during the inflation process. As air escapes through these small holes, a thin air cushion is formed between the device and the surgical table, enabling the frictionless movement of the patient. This hover effect is a key component of the lateral patient transfer function, allowing medical personnel to safely transfer the patient with minimal physical effort.

In an embodiment of the present invention, the device incorporates flexible padded substrates attached to its top surface. These substrates are designed to wrap around the patient's limbs, creating a secure wrapped engagement that holds the patient's arms and legs in place during surgery. The padding helps to protect vulnerable areas, such as the ulnar nerve, from excessive pressure, thereby minimizing the risk of nerve damage or pressure ulcers. The slight elevation of the limbs above the surgical table surface further reduces the risk of injury. By securing the patient's limbs, the device prevents the patient from shifting or sliding off the table, particularly while under anesthesia. This is critical in ensuring the patient's safety throughout the procedure. The Velcro straps, buckles, and webbing are strategically placed to fasten both the device and the patient securely to the surgical table. These securements ensure that the device remains stable, even when the operating table is adjusted (e.g., tilting or rotating).

A significant feature of the device is its ability to regulate the patient's body temperature during surgery, helping to prevent hypothermia, which is a common concern in the operating room due to exposure to cold environments and anesthesia.

The three-layer warming blanket (composed of the first, second, and third layers) works with a forced-air warming machine to circulate warm air around the patient. Air is introduced between the second and third layers (chamber #2) and circulates through channels formed by stitched lines within the blanket. These channels direct warm air toward the patient, which then escapes through the 1 mm holes in the second layer and finally through the micro-perforations in the top layer.

The fifth layered structure ensures that warm air is distributed evenly, with no hotspots or areas of stagnation. The periphery of the warming blanket is sealed using double-sewn lines or heat-sealing technology to prevent air from escaping at the edges, maximizing the efficiency of the warming system.

After surgery, the patient often needs to be transferred from the operating table to a transport bed. Traditional methods of manually lifting or sliding the patient can be cumbersome and risk injury to both the patient and healthcare staff. The present invention addresses this issue by incorporating inflatable support and hover technology.

The bottom two layers of the device (fourth and fifth layers) are designed to inflate when connected to a high-flow air machine. During inflation, air is trapped in the fourth layer, slightly elevating the patient above the table surface. Air then escapes through small holes in the fifth layer, creating a cushion of air that reduces friction between the device and the table. This air cushion enables the frictionless transfer of the patient from one surface to another, reducing the need for physical lifting and minimizing the risk of injury.

To prevent the pad from shifting during surgery, inflation, or lateral transfer, the invention incorporates multiple anti-slip mechanisms. In an embodiment of the present invention, thin non-slip strips are integrated into the bottom layer of the mat. These strips provide frictional resistance, preventing the pad from moving when it is deflated and the patient is positioned on it. During the inflation process, strategically placed holes in the bottom layer generate a cushion of air around the non-slip strips. This air cushion neutralizes the friction created by the strips, allowing for smooth patient transfer without compromising the non-slip function when the pad is deflated. The device is equipped with fasteners and straps to anchor it to the surgical table, preventing unwanted sliding during surgery, particularly when the table is adjusted to different angles (e.g., head-down or lateral-tilt positions).

The elevated strips are constructed from durable materials such as rubber, polymer, or thick tape or integrally formed by sewn folds in the fabric during manufacturing or equivalent methods, range in thickness from 0.5 mm to 5 mm and are positioned longitudinally to retain escaped air beneath the mattress, these strips are affixed longitudinally along the sides of the mattresses. They range in thickness from approximately 0.5 mm to 5 mm and extend along most of the device's length. These strips act as barriers to lateral air escape, retaining the air cushion beneath the device during inflation. Their low profile ensures minimal interference with lateral transfer or patient positioning.

During inflation, the elevated strips significantly improve the air cushion's stability by preventing lateral dissipation of escaped air. The retained air beneath the mattress creates a consistent, robust cushion that facilitates smooth and efficient patient transfers. By reducing air loss, the strips also decrease the operational demands on the air supply system, enhancing energy efficiency and minimizing noise.

The operation of the patient positioning, warming, and lateral transfer device is simple yet highly effective, combining various functions into a unified system to ensure patient safety and comfort throughout a surgical procedure. The process is broken down into three main stages: pre-surgery setup, during surgery, and post-surgery patient transfer.

The device is first laid out flat on the operating table. The bottom side of the device, equipped with non-slip strips, is placed directly onto the surface of the table to prevent unwanted movement during patient positioning and surgery. The patient is carefully placed on top of the device. The patient's head, torso, and limbs are positioned in line with the designated areas on the pad. The perforations and padding structures on the device are located to support critical areas like the head, arms, legs, and upper torso.

The padded substrates, positioned on the device's surface, are wrapped around the patient's arms and legs using Velcro straps, buckles, or webbing to stabilize the limbs to prevent any shifting or unwanted movement during the procedure. The substrates also protect the patient's skin and nerves, particularly in areas like the arms where the ulnar nerve is sensitive to pressure.

A forced-air warming machine is connected to the device via the air inlet located at the sides or corners of the mat. This air inlet is designed to accommodate various air hoses and can be secured tightly using elastic bands to ensure a snug fit. The warming system is activated at this point, but warm air will not yet flow until the surgery begins.

During surgery, the patient remains securely positioned due to the wrapping engagements around their limbs. The Velcro straps and fasteners prevent any lateral or rotational movement of the patient, ensuring the patient stays in the proper surgical position throughout the procedure. The securement system also helps prevent patient sliding or displacement if the surgical table is tilted or adjusted.

Once the procedure begins, the forced-air warming machine is turned on to regulate the patient's body temperature. The air inlet allows warm air to flow between the second and third layers of the device, which forms a warming blanket. This warm air circulates through channels created by stitched lines inside the blanket, ensuring that the heat is evenly distributed across the patient's body. The 1 mm perforations in the second layer direct the warm air upwards, allowing it to pass through the top layer's micro-perforations to reach the patient. The fluid-resistant top layer ensures that the patient's body remains dry by preventing blood or other liquids from penetrating the lower layers of the device.

During surgery, medical tubes such as IV lines or catheters can be securely fastened to the device using built-in Velcro straps and webbing. This feature prevents the lines from becoming tangled or accidentally dislodged, ensuring uninterrupted delivery of medications and fluids to the patient.

At the end of the surgical procedure, the warming system is turned off, and the air hose is disconnected from the air inlet. The device remains under the patient as they are prepared for transfer to a recovery bed. For lateral transfer, the device is equipped with a lateral transfer feature based on hover technology. A high-flow air machine is connected to the air inlet located in the bottom layers of the mat and then the air machine is turned on, inflating the fourth layer (air chamber) of the device. As the fourth layer inflates, it slightly lifts the patient from the surface of the operating table, creating an air cushion beneath the patient. Once inflated, air escapes through small perforations in the bottom fifth layer, creating a thin layer of air between the device and the surgical table.

This air cushion reduces the friction between the patient and the table surface, effectively allowing the patient to “hover” slightly above the table. This frictionless state enables medical personnel to easily slide the patient from the operating table to a transport bed. With the air cushion in place, medical staff can gently push the patient from the surgical table to a recovery bed or stretcher. The hover technology reduces the physical effort required to move the patient and minimizes the risk of injury both to the patient and to the healthcare providers. After the patient has been successfully transferred, the high-flow air machine is turned off, and the device deflates quickly. The device can then be easily removed from beneath the patient, leaving the patient securely positioned on the recovery bed.

While the exemplary embodiments of the present invention are described and illustrated herein, it will be appreciated that they are merely illustrative. It will be understood by those skilled in the art that various modifications in form and detail may be made therein without departing from or offending the spirit and scope of the invention as defined by the appended claims. Additionally, the invention illustratively disclosed herein suitably may be practiced in the absence of any element which is not specifically disclosed herein-and in particular embodiment specifically contemplated, is intended to be practiced in the absence of any element which is not specifically disclosed herein.

1. A patient positioning, warming, and lateral transfer device, comprising: a flexible sheet configured to be placed on a support surface, such as a surgical table or a hospital bed; a first and second flexible substrate coupled to the top surface of the sheet, wherein the substrates are padded and capable of wrapping around a patient's arms and/or legs, creating a wrapped engagement to protect, pad, secure, and elevate the arms and/or legs of the patient; a temperature-regulating pad incorporated into the device, comprising an air-permeable top layer, a perforated middle layer with air channels, and a bottom layer; multiple hook-and-loop fasteners, Velcro straps, and webbing with buckles at various locations to secure the patient to the support surface and stabilize critical medical lines or tubes; an inflatable support system integrated within the bottom layers of the device, facilitating patient transfer by reducing friction between the patient and the support surface when inflated; and two or more elevated strips positioned longitudinally along the sides of the mattress, wherein the strips are configured to retain escaped air beneath the mattress during inflation.

2. The patient positioning, warming, and lateral transfer device of claim 1, wherein the temperature-regulating pad is configured to deliver warm air to the patient, comprising: an air inlet for receiving forced warm air from an external air-warming machine; a top layer made of fluid-resistant, air-permeable material to discharge warm air into the patient's airspace through microscopic perforations;
a perforated middle layer with strategically positioned holes to distribute warm air across key regions of the patient's body, including the arms, legs, and torso; and
a bottom layer defining an air chamber between the middle and bottom layers for circulating warm air through sewn channels, ensuring uniform heat distribution.

3. The patient positioning, warming, and lateral transfer device of claim 2, wherein the edges of the warming pad are sealed using a double-sew line and heat-sealing technology to prevent heat loss from the blanket and to enhance warming efficiency.

4. The patient positioning, warming, and lateral transfer device of claim 1, further comprising: a fourth layer without holes and a fifth layer with holes positioned at the bottom of the device; a high-flow air machine connectable to the fourth and fifth layers to inflate the layers, creating an air cushion between the device and the support surface; small perforations in the fifth layer allowing air to escape, thereby creating a low-friction environment between the patient and the surgical table for lateral patient transfer.

5. The patient positioning, warming, and lateral transfer device of claim 4, wherein the air cushion enables lateral transfer of the patient by reducing friction between the device and the support surface, facilitating smooth patient movement from the surgical table to a transport bed.

6. The patient positioning, warming, and lateral transfer device of claim 1, wherein the padded substrates used for wrapped engagement are configured to secure the patient's limbs, specifically the arms and legs, to prevent unintentional movement during surgery, and elevate the patient's limbs slightly off the surgical table surface to minimize pressure injuries to sensitive nerve areas, such as the ulnar nerves.

7. The patient positioning, warming, and lateral transfer device of claim 1, wherein the Velcro straps, webbing, and buckles are strategically placed on the device to secure medical tubes and critical lines attached to the patient during surgery, and secure the device to the surgical table, preventing the patient and device from shifting or sliding during surgery, particularly during table tilting or other positional adjustments.

8. The patient positioning, warming, and lateral transfer device of claim 1, further comprising non-slip strips integrated into the bottom layer of the device, providing frictional resistance to prevent the device from sliding during surgery when not inflated, and a mechanism to neutralize the effect of the non-slip strips during patient transfer by activating the hover technology, wherein air holes in the bottom layer create a thin air cushion, allowing for smooth lateral transfer.

9. The patient positioning, warming, and lateral transfer device of claim 4, wherein the inflatable support system creates a controlled inflation process, such that the lateral sections of the device inflate first, followed by the central section supporting the patient's body weight, resulting in a trough-like formation that cradles the patient and secures them during inflation and transfer.

10. The patient positioning, warming, and lateral transfer device of claim 9, wherein the sequential inflation of lateral and central section ensures patient safety by minimizing the risk of patient displacement or sliding during transfer.

11. The patient positioning, warming, and lateral transfer device of claim 1, wherein the wrapped engagements, Velcro straps, and padded substrates are configured to maintain the patient's secure positioning on the surgical table, preventing any accidental sliding or movement during anesthesia, and provide extra stability to prevent patient displacement during the inflation of the device when preparing for lateral transfer.

12. The patient positioning, warming, and lateral transfer device of claim 9, wherein the inflatable support system, when fully inflated, causes the sides of the device to rise higher than the central section, creating a cradling effect that secures the patient during transfer.

13. The patient positioning, warming, and lateral transfer device of claim 2, wherein the temperature-regulating pad ensures patient comfort by: dispersing warm air evenly across the surface of the device, creating a soft, consistent air cushion that maintains the patient's body temperature during surgery; ensuring warm air circulates within air chambers and exits through the microscopic perforations on the top layer, creating a consistent temperature in the patient's airspace.

14. The patient positioning, warming, and lateral transfer device of claim 2, wherein the sewn lines within the warming system prevent air stagnation and ensure uniform heat distribution, avoiding the formation of hot spots across the device.

15. The patient positioning, warming, and lateral transfer device of claim 2, wherein the air inlet is universally compatible with various types of forced-air warming machines, secured by elastic bands to fit different air hoses.

16. The patient positioning, warming, and lateral transfer device of claim 1, wherein the multiple layers of the device, including the warming pad and lateral transfer system, are sewn or adhesively bonded to form a unitary structure, providing durability and reliability during use.

17. The patient positioning, warming, and lateral transfer device of claim 1, wherein the device is equipped with fastening mechanisms on both sides that can be attached to the rails of the surgical table, preventing displacement of the device and ensuring patient safety even when the table is tilted or adjusted during surgery.

18. The patient positioning, warming, and lateral transfer device of claim 1, wherein the elevated strip constructed from rubber, polymer, thick adhesive tape or integrally formed by sewn folds in the fabric during manufacturing or equivalent methods have thickness ranging from 0.5 mm to 5 mm.
